(19) United States
(12) Patent Application Publication
(10) Pub. No.: US 2024/0131122 A1
(43) Pub. Date: Apr. 25, 2024

(54) SITE-SPECIFIC CLEAVAGE AND ELIMINATION OF DNA IN BACTERIAL SPECIES WITH SEGMENTED CHROMOSOMES

(71) Applicants: Brandeis University, Waltham, MA (US); The Board of Trustees of the Leland Stanford Junior University Leland Stanford Junior University JUN, Palo Alto, CA (US)

(72) Inventors: Yuko Nakajima, Lincoln, MA (US); James E. Haber, Wayland, MA (US); Christine Jacobs-Wagner, Emerald Hills, CA (US); Constantin N. Takacs, Medford, MA (US)

(21) Appl. No.: 18/469,664

(22) Filed: Sep. 18, 2023

Related U.S. Application Data
(60) Provisional application No. 63/407,955, filed on Sep. 19, 2022.

(51) Int. Cl. A61K 38/46; C12N 9/22; C12N 15/11; C12N 15/74

(52) U.S. Cl. CPC A61K 38/465; C12N 9/22; C12N 15/11; C12N 15/74; C12N 2310/20; C12N 2800/101; C12N 2800/80; C12N 2830/002

(57) ABSTRACT

Described herein is a method of reducing virulence of a bacterial species with a segmented genome which has infected a mammalian host or mammalian host cell, by exposing the bacterial genome to an RNA-guided nuclease and a guide RNA (gRNA), thus generating a double-stranded or single-stranded break in the bacterial genome and causing loss of the targeted genome segment.

### CROSS-REFERENCE TO RELATED APPLICATIONS

This application claims priority to U.S. Provisional Application 63/407,955 filed on Sep. 19, 2022, which is incorporated herein by reference in its entirety.

### STATEMENT REGARDING FEDERALLY SPONSORED RESEARCH & DEVELOPMENT

This invention was made with government support under GM127029 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on Sep. 9, 2023 is named SEQ_LIST107648 002 and is 47.7 KB in size. The Sequence Listing does not go beyond the disclosure in the application as filed.

### BACKGROUND

Lyme disease, also known as Lyme borreliosis, is the most prevalent vector-borne disease in North America and Eurasia. It is caused primarily by the spirochete Borrelia burgdorferi and the related Borrelia afzelii and Borrelia garinii species. The disease presents with various symptoms that can include fever, malaise, rash, arthritis, neurological dysfunctions, and cardiac manifestations. Humans are accidental hosts. In nature, B. burgdorferi is typically maintained through a transmission cycle between a vertebrate host reservoir (e.g., white footed mice and other small mammals, but also birds) and an ixodid tick vector. During feeding, B. burgdorferi-colonized tick vectors deliver the spirochetes into vertebrate hosts, where the spirochetes can replicate, disseminate, and often establish persistent infection.

Members of the Borreliaceae family contain the most segmented bacterial genomes known to date. For instance, the genome of the B. burgdorferi type strain B31 is composed of a linear chromosome and 21 linear and circular plasmids. During growth in culture under abundant nutrient condition, the Borreliaceae could be polyploid with each cell carrying multiple copies of both the chromosome and plasmids. The chromosome encodes the vast majority of essential housekeeping and metabolic functions. In contrast, the plasmids primarily encode lipoproteins that mediate the spirochetes' interaction with the vertebrate and tick host environments and help them evade host immune defenses. Additionally, each strain hosts several highly similar plasmid members of the cp32 class, which are prophages. In the B. burgdorferi type strain B31, which is the most well studied genetically, only plasmid cp26 has been shown to be required for growth in axenic culture. Several other plasmids are known to be required in the vertebrate or tick hosts. However, much remains unknown about the roles of B. burgdorferi plasmids. Furthermore, as the number of distinct plasmid types and the genes carried by any given plasmid type vary significantly among Borreliaceae species and strains, strain-to-strain inferences of plasmid function are not always possible.

An effective way to investigate plasmid function is to remove it from a given strain. Spontaneous plasmid loss during extended passaging in axenic culture has been known since the early days of Lyme disease research, but this approach is not specific to a particular plasmid of interest and often results in loss of multiple plasmids. Curing a specific plasmid can be achieved through transformation of B. burgdorferi with a shuttle vector that carries the plasmid maintenance locus of the endogenous plasmid of interest. The incompatibility that arises between the endogenous plasmid and the introduced shuttle vector leads to displacement of the endogenous plasmid by the shuttle vector. However, this approach requires knowledge of the plasmid maintenance locus of the targeted endogenous plasmids.

What is needed are new methods of cleaving genomic DNA such as endogenous virulence plasmids in a bacterial species with a segmented genome such as a Borrelia sp. Such methods could provide an alternative or supplement to antibiotic therapy which is generally less than fully effective and can cause severe side effects.

### BRIEF SUMMARY

In one aspect, a method of reducing virulence of a bacterial species with a segmented genome which has infected a mammalian host or mammalian host cell comprises exposing the bacterial genome to an RNA-guided nuclease and a guide RNA (gRNA) and generating a double-stranded or single-stranded break in the bacterial genome, wherein the gRNA base pairs with a target sequence in the bacterial genome. In an aspect, the target sequence in the bacterial genome is in a bacterial chromosome or an endogenous virulence plasmid, such as an endogenous virulence plasmid.

### BRIEF DESCRIPTION OF THE DRAWINGS

FIG. 1 is a schematic depiction of E. coli/B. burgdorferi shuttle vectors. Left, all-in-one, Cas9-targeting shuttle vectors carrying a single-guide RNA (sgRNA) expression cassette as well as an IPTG-inducible Cas9 expression cassette that contains a constitutively expressed lad gene. Right, non-targeting Cas9 shuttle vectors, which lack the sgRNA cassette. The Cas9 versions used are, from top to bottom: dCas9, Cas9^{D10A}, Cas9^{H840A}, and Cas9^{WT}. The presence of the D10A or H840A mutation is indicated by arrowheads. Features are not drawn to scale. For simplicity, other important features of the shuttle vectors, such as the antibiotic resistance cassette or the E. coli or B. burgdorferi origins of replication, are not marked on the figure.

FIGS. 2A-C show locations targeted by Cas9 activity in B. burgdorferi endogenous plasmids lp28-1 and lp25. 2A. Top: schematic depiction of plasmid lp28-1. Marked (but not drawn to scale) are gene bbf03 and the vls locus, which were targeted by the indicated sgRNAs. The sgRNAs were used one at a time, never in combination. Middle: magnification of the vls locus. Shown (but not drawn to scale) are the expressed vlsE lipoprotein gene and the 15 silent vls cassettes. Bottom: magnified view of the vlsE1 cassette, which contains the variable regions of the expressed vlsE lipoprotein, flanked by two direct repeats (DRs). Variable regions (VRs) 1 through 6 are depicted, as well as the locations targeted by sgRNAvlsE1 and sgRNAvlsE2. Covalently closed hairpin telomeres are depicted as ovals flanking both ends of the linear plasmid. 2B. Same as in (A) but for plasmid lp25. Marked (but not drawn to scale) are genes bbe10 and bbe17, which were independently targeted by the indicated sgRNAs. 2C. Depiction of part of the vlsE gene of strain K2. This strain was recovered from an infected mouse and contains segments of vls5, vls6, vls8 (or vls16) and vls14, indicating that there were at least four recombination events during strain construction. Shown in shades of gray are sequences shared with the vlsE sequence reported for the parental strain B31. In shade of grey are divergent sequences that likely arose by recombination of the indicated silent cassettes into the expressed locus. The shade of grey match those used for the silent cassettes in panel A. The vls8116 notation signifies a sequence that could have originated from either the vls8 or vls16 silent cassette.

FIG. 3 shows targeting Cas9 activity to lp28-1 causes the loss of this plasmid from the cell population. Cells of B. burgdorferi strain K2 were electroporated with shuttle vectors expressing Cas9^{WT} and the indicated sgRNAs. Four clones obtained from each transformation were analyzed by multiplex PCR for the presence or absence of B. burgdorferi endogenous plasmids. The PCR reactions were grouped into six sets. The endogenous plasmids corresponding to each of the bands are listed on the right. Signal intensity was scaled to ensure that all positive bands could be seen. As a result, the intensities of some bands are saturated.

FIG. 4 shows a summary of the B. burgdorferi transformation results. Graph compiling the plasmid retention values measured in experiment 3 described in Tables 4 and 5. Plasmid retention was calculated by dividing the concentration of cells that received the Cas9/sgRNA-expressing shuttle vector and retained the targeted plasmid by the concentration of cells that received the shuttle vector for any given electroporation. Experimental samples were grouped as follows. The “No sgRNA” group combines transformations of shuttle vectors encoding each of the four Cas9 versions (Cas9^{WT}, Cas9^{D10A}, cas9^{H840A}, and dCas9) but no sgRNA. These transformations were plated under either kanamycin or gentamicin selection to assess retention of lp25 or lp28-1, respectively. All other transformations are grouped based on the version of Cas9 expressed from the shuttle vector and combine lp28-1 and lp25-targeting constructs.

FIGS. 5A-C show transformation statistics. 5A. Transformation yield, defined as number of shuttle vector-transformed colony forming units (cfu) per mL culture measured by plating under streptomycin selection one day after electroporation. The experiment numbers are the same as in Tables 4 and 5 and are listed at the bottom. Recipient strains are also listed at the bottom. 5B. Plot showing the distribution of transformation frequency values measured in experiment 3 described in (A) and Tables 4 and 5. The transformation frequency is defined as the fraction of the number of cells that got transformed with the Cas9 shuttle vector to the number of cells that survived electroporation. Transformed and viable cell numbers were measured by plating the electroporated cells in the presence or absence of streptomycin. 5C. Plot showing the limit of detection of cells that retained the targeted endogenous plasmid for the individual electroporations performed in experiments 1 through 3 as described in panel (A) and Tables 4 and 5. The limit of detection values were calculated as follows. First, we counted the number of colonies obtained by plating the electroporated B. burgdorferi cultures under streptomycin selection and added the values we counted on the plates seeded with the 100, 300, and 900 μL cell culture volumes (1.3 mL total, also see Methods). If plates seeded with larger volumes of culture yielded too many colonies to allow an accurate count, the number of colony-forming cells in 1.3 mL culture was estimated from the number of colonies counted on plates seeded with the lesser culture volume(s). The limit of detection value was then calculated as the inverse of the number of colonies obtained from plating 1.3 mL electroporated culture. Labels are as in (A).

FIG. 6A shows the new construct with conditional promoters for both Cas9 and sgRNA. ori: E. coli origin of replication; PflgB: B. burgdorferi flgB promoter; PflaB: B. burgdorferi B31 flaB promoter; IR1-PF57-PF50-PF49-IR2: sequences required for B. burgdorferi replication and plasmid maintenance; pQE300: two copies of E. coli Lac operator inserted in a synthetic promoter; pflac: an E. coli Lac operator inserted within the B. burgdorferi flaB promoter; gRNA spacer-Cas9 binding region-terminator1: constitutes a single-guide RNA. SmR: Streptomycin/Spectinomycin resistance gene.

FIG. 6B shows that dual regulation of sgRNA and Cas9 expression allows rapid and sensitive elimination of the targeted plasmids in culture. While catalytically inactive Cas9 (dCas9) shows no effect on the targeted plasmid abundance (left), wild-type Cas9 selectively eliminates the targeted plasmid within 20 hours in culture at the lowest concentration of IPTG (right).

The above-described and other features will be appreciated and understood by those skilled in the art from the following detailed description, drawings, and appended claims.

### DETAILED DESCRIPTION

As described herein, in one aspect, the inventors determined a method to eliminate endogenous plasmids from B. burgdorferi strains would be to generate site-specific DNA lesions. In the absence of efficient DNA repair, those lesions were predicted to lead to degradation of the targeted endogenous plasmid. In the absence of a recombinational donor sequence, exogenously induced double-stranded DNA breaks (DSB s) in the chromosome can be lethal in several bacteria, including Escherichia coli, streptococci, Clostridium cellulolyticum, and the spirochete Leptospira biflexa. Repair of a site-specific DSB in Neisseria gonorrhoeae, when there are no homologous sequences to provide a template for recombinational repair, occurs at such low frequencies that less than one cell in ten thousands survives this type of genome lesion. In contrast, the presence of short (5 to 23 base pairs) homologous sequences flanking an endonuclease-induced DSB led to RecA-mediated repair in a small fraction of cells. Since most B. burgdorferi plasmids are not needed for growth in axenic culture, induction of DNA lesions in B. burgdorferi plasmids should cause plasmid loss if DNA repair is inefficient.

As described herein, to generate such site-specific lesions, the clustered regularly interspaced palindromic repeats (CRISPR)-Cas9 system derived from Streptococcus pyogenes was employed. Cas9 is the endonuclease component of a type of bacterial innate immunity defense against invading foreign DNA molecules. It has two catalytic residues, D10 and H840, each cutting one of the strands of the targeted double stranded DNA sequence. Cas9 targeting to a specific DNA sequence can be achieved by co-expression of a short guide RNA molecule, or gRNA. Base pairing between the Cas9-bound sgRNA and the target DNA sequence next to a protospacer-adjacent motif (PAM) directs the Cas9 activity to the genome location specified by the sgRNA. While wild-type Cas9 (Cas9^{WT}) generates a DSB in the target DNA sequence, single active site mutants (Cas9^{D10A} and Cas9^{H840A}) are nickases that generate single-stranded DNA breaks (SSBs). Finally, the double mutant, catalytically dead Cas9 D10A/H840A, or dCas9, does not create DNA lesions and thus serves as a negative control. dCas9, however, can interfere with transcription when targeted to promoters and promoter-proximal coding region. A dCas9-based CRISPR interference (CRISPRi) platform in B. burgdorferi has been previously reported. Described herein are the effects of targeting Cas9^{WT} and its nickase versions to several B. burgdorferi endogenous plasmid loci.

In an aspect, method of reducing virulence of a bacterial species with a segmented genome which has infected a mammalian host or mammalian host cell comprises exposing the bacterial genome to an RNA-guided nuclease and a guide RNA (gRNA) and generating a double-stranded or single-stranded break in the bacterial genome, wherein the gRNA base pairs with a target sequence in the bacterial genome. In an aspect, the target sequence in the bacterial genome is in a bacterial chromosome or an endogenous virulence plasmid. For example, the genome of B. burgdorferi type strain B31 is composed of a linear chromosome and 21 linear and circular plasmids.

As used herein, a bacterial species with a segmented genome is a bacterial species in which the genome is segmented into several different pieces of genetic material instead of being on one piece of DNA. Segmented genomes can include circular and linear chromosomes, chromids, megaplasmids, as well as smaller plasmids. Exemplary bacterial species with segmented genomes include Borrelia sp., Vibrio sp., Agrobacteria sp., Bacillus sp., Brucella sp., Burkholderia sp., Leptospira sp., Rhizobium sp., and Rhodobacteria sp. Exemplary Borrelia sp. include Borrelia burgdorferi, Borrelia afzelii, Borrelia garinii, Borrelia hermsii, Borrelia turicatae, and Borrelia parkeri. Translation of the methods described herein across bacterial phyla is expected due to the successful implementation of CRISPR-based methods across species.

In an aspect, reducing virulence of a bacterial species comprises targeting a virulence plasmid of the bacterial species. Virulence plasmids are plasmids inside of a bacterium that comprise virulence determinants which enhance the pathogenicity of the bacterium. In the case of Borrelia burgdorferi, virulence plasmids include the lp25, lp28-1, lp36 and cp32 plasmids. Exemplary target genes on lp25 include bbe10 and bbe17. Exemplary target genes on lp28-1 include the vlsE lipoprotein gene, a vls2-vls16 silent cassette, or a non-vls locus, such as bbf03.

The method includes targeting the genome or a bacterial virulence plasmid of the bacterial species with an RNA-guided nuclease and a guide RNA (gRNA) to generate a double-stranded or single-stranded break in the genome or bacterial virulence plasmid. RNA-guided nucleases include, but are not limited to, Class 2 CRISPR nucleases such as Cas9 and Cpf1, as well as other nucleases derived or obtained therefrom. As used herein, the terms Cas9 and Cpf1 include active derivatives of these RNA-guided nucleases. RNA-guided nucleases are defined as those nucleases that: (a) interact with (e.g., complex with) a gRNA; and (b) together with the gRNA, associate with, to cleave or modify, a target region of a DNA that includes (i) a sequence complementary to the targeting domain of the gRNA and, typically, (ii) an additional sequence referred to as a “protospacer adjacent motif,” or “PAM”. RNA-guided nucleases can be defined, in broad terms, by their PAM specificity and cleavage activity, even though variations may exist between individual RNA-guided nucleases that share the same PAM specificity or cleavage activity.

Various RNA-guided nucleases may require different sequence and spatial relationships between PAMs and protospacers. In general, Cas9 nucleases recognize PAM sequences that are 3′ of the protospacer. Cpf1 generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT (R is A or G and N is any nucleotide) or NNGRRV (V is G, C or A), wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described in the art. It should also be noted that engineered RNA-guided nucleases can have PAM specificities that differ from the PAM specificities of reference molecules.

Examples of polypeptide sequences of Cas9 molecules that may be used according to the embodiments herein are set forth in SEQ ID NOs: 29 (Streptococcus mutans), 30 (Streptococcus pyogenes), 31 (Streptococcus thermophilus), 32 (Listeria innocua), 33 (Staphylococcus aureus), and 34 (Neisseria meningitidis). In certain embodiments, the Cpf1 protein may comprise a sequence selected from the group consisting of SEQ ID NOs. 35 (Acidaminococcus sp. strain BV3L6), 36 (Lachnospiraceae bacterium ND2006), and 37 (Lachnospiraceae bacterium MA2020).

In an aspect, wild-type Cas9 (Cas9^{WT}) generates a DSB in the target DNA sequence. Single active site mutants (Cas9^{D10A} and Cas9^{H840A}) are nickases that generate single-stranded DNA breaks (SSBs). Any of the foregoing Cas9 polypeptides can be used in the methods described herein.

Also included are polynucleotides encoding the RNA-guided nuclease. The polynucleotide can include operably linked nucleotide regulatory sequences (i.e., non-coding sequences) that produce a functional gene. Thus, an RNA-guided nuclease ORF (open reading frame) can be operably linked to regulatory sequences that lead to accurate and efficient transcription and translation of the ORF. These regulatory sequences can include, but are not limited to, enhancer elements, promoter elements, termination sequences (for either transcription or translation), polyadenylation signal sequences and intron/exon splicing sequences. In some embodiments, a polynucleotide encoding the RNA-guided nuclease is contained in an expression vector, which may have other nucleotide sequences including an origin of replication, one or more selectable markers, a visual marker, and/or a site that facilitates manipulation of the vector and insertion or subcloning of additional gene sequences, such as one or more restriction sites or a multiple cloning site (MCS).

In the methods described herein, the RNA-guided nuclease may be delivered as a polynucleotide expressing the RNA-guided nuclease ORF, or as a polypeptide. If the RNA-guided nuclease is delivered as a polynucleotide expressing the RNA-guided nuclease ORF, the polypeptide is expressed after delivery to a cell capable of expressing the ORF.

The terms “guide RNA” and “gRNA” refer to a nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric or as a single-guide RNA (sgRNA)), or modular (comprising more than one, and typically two, separate RNA molecules, such as a crRNA and a tracrRNA, which are usually associated with one another, for instance by duplexing).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences”, “complementarity regions”, “spacers” and generically as “crRNAs”. Targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For example, the duplex structure formed by first and secondary complementarity domains of a gRNA (also referred to as a repeat:anti-repeat duplex) interacts with the recognition (REC) lobe of Cas9 and can mediate the formation of Cas9/gRNA complexes.

In an aspect, an RNA-guided nuclease binds a guide RNA and is targeted to a specific sequence (a target site) in a target nucleic acid. An RNA-guided nuclease is targeted at a target site by the Cas9 guide RNA to which it is bound. The guide RNA comprises a sequence that is complementary to a target sequence within the target nucleic acid, thus targeting the bound RNA-guided nuclease to a specific location within the target nucleic acid (the target sequence) (e.g., stabilizing the interaction of RNA-guided nuclease with the target nucleic acid).

As used herein, when the bacterial virulence plasmid is the lp25 plasmid, the spacer sequence of the guide RNA may comprise SEQ ID NO: 1 or SEQ ID NO: 2. When the bacterial virulence plasmid is the lp28-1 plasmid, the spacer sequence of the guide RNA may comprise any of SEQ ID NOs. 3-6. The gRNA may comprise any sequence adjacent to a PAM that is not found elsewhere in the target genome.

The gRNA can be delivered in the form of an expression cassette or as RNA. RNA can be prepared in an in vitro transcription reaction. In some embodiments, generating in vitro transcribed RNA comprises incubating a linear DNA template with an RNA polymerase and a nucleotide mixture under conditions to allow (run-off) RNA in vitro transcription. The nucleotide mixture can be part of an in vitro transcription mix (IVT-mix). In some embodiments, the RNA polymerase is a T7 RNA polymerase. In some embodiments, the nucleotide mixture is composed of (chemically) non-modified ribonucleoside triphosphates (NTPs) GTP, ATP, CTP and UTP. In some embodiments, the in vitro transcription can include the presence of modified nucleotides as is known in the art.

In an aspect, a gene encoding the RNA-guided nuclease and an expression cassette for the gRNA are carried on a vector comprising the expression cassette for expressing the gRNA and an inducible expression cassette for expressing the RNA-guided nuclease. In an aspect the vector is a shuttle vector that can be replicated in E. coli and the bacterial species with a segmented genome. Shuttle vectors are replicating DNA plasmids that can express both the RNA-guided nuclease and gRNA in the host bacterial strain.

In another aspect, an inducible expression cassette for expressing the RNA-guided nuclease and an expression cassette for the gRNA are encoded in the DNA of a bacteriophage. The term “phage” is interchangeable with the term “bacteriophage” and refers to a virus that infects bacterial cells. Phages include an outer protein capsid enclosing genetic material. The genetic material can be ssRNA, dsRNA, ssDNA, or dsDNA, in either linear or circular form. Phages and phage vectors are well known to those of skill in the art and non-limiting examples of phages are λ (Lysogen), T2, T4, T7, T12, R17, M13, MS2, G4, P1, P2, P4, Phi X174, N4, Φ6, and Φ29. Bacteriophages allow efficient delivery of DNA sequence-specific RNA-guided nuclease antimicrobials into bacteria in vivo as a therapeutic option to treat bacterial infections, for example.

In another aspect, the RNA-guided nuclease and gRNA can be packaged and delivered in a liposome or nanoparticle, such as a lipid nanoparticle (LNP), in the form of plasmid DNA or in vitro transcribed RNA.

In another aspect, the pre-assembled RNA-guided nuclease and gRNA are formulated in a liposome or nanoparticle such as a LNP.

In another aspect, the RNA-guided nuclease and gRNA can be packaged and delivered in extracellular vesicles (EVs).

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Polynucleotides associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid.

A LNP is a non-viral delivery system that safely and effectively delivers nucleic acids to a target. The term “lipid nanoparticle” refers to a nanoscopic particle composed of lipids having a size measured in nanometers (e.g., 1-5,000 nm). In some embodiments, the lipids comprised in the lipid nanoparticles comprise cationic lipids and/or ionizable lipids. Cationic lipids and/or ionizable lipids known in the art can be used to formulate LNPs for delivery of gRNA and RNA-guided nuclease to the cells. Exemplary cationic lipids include one or more amine group(s) bearing positive charge. In some embodiments, the cationic lipids are ionizable such that they can exist in a positively charged or neutral from depending on pH. In some embodiments, the cationic lipid of the lipid nanoparticle comprises a protonatable tertiary amine head group that shows positive charge at low pH. The lipid nanoparticles can further comprise one or more neutral lipids (e.g., Di stearoylphosphatidylcholine (DSPC), 1,2-Dioleoyl-sn-glycero-3-phosphocholine (DOPC), 1,2-Dimyristoyl-sn-glycero-3-phosphoethanolamine (DMPE), 1,2-Dipalmitoyl-sn-glycero-3-phosphorylethanolamine (DPPE) etc. as a helper lipid), charged lipids, steroids, and polymers conjugated lipids. In some embodiments, the LNP can comprise cholesterol. In some embodiments, the LNP can comprise a polyethylene glycol (PEG) lipid.

In some embodiments, the molar percent of an ionizable lipid in the total lipid of a lipid nanoparticle is about, at least, at least about, at most or at most about 30%, 35%, 40%, 45%, 50%, 55%, 60%, 65%, 70%, or a number or range between any two of these values. In some embodiments, the molar percent of an ionizable lipid in a lipid nanoparticle is in a range between about 40-70% (e.g., about 60%). In some embodiments, the lipid nanoparticle can further comprise a helper lipid (e.g., DSPC), a sterol lipid (e.g., cholesterol), and PEG lipid or a phospholipid PEG conjugate. In some embodiments, the molar percent of a helper lipid in a lipid nanoparticle is about 5%-20% (e.g., about 10.5%), the molar percent of a sterol lipid is about 10%-40% (e.g., about 21%), and the molar percent of a PEG lipid is about 0.5%-10% (e.g., about 8.5%).

EVs can be generated by organisms and function in cell-to-cell communication by transferring biomacromolecules (e.g. nucleic acids, proteins, lipids, glycoproteins). Borrelia sp. generate EVs that contains biomacromolecules and methods for EV isolation from Borrelia have been documented.

In an aspect, exposing the bacterial genome comprises administering the RNA-guided nuclease and gRNA to a mammalian subject. In an aspect, the mammal is a human, or a rodent species, for example.

Advantageously, the treatment method described herein is expected to be effective in all stages of infection. For example, in Lyme disease, stage one is localized Lyme disease in which the bacteria have not yet spread throughout the body. Stage 2 is early disseminated Lyme disease in which the bacteria have begun to spread through the body. And stage 3 is late disseminated Lyme disease in which the bacteria have spread to more distant sites such as joints and nerves.

The treatment described herein can be combined with administration of an antibiotic such as doxycycline, amoxicillin, cefuroxime, ceftriaxone, hygromycin A, and the like, and combinations thereof.

The invention is further illustrated by the following non-limiting examples.

### Examples

### Methods

E. coli strains and growth conditions: E. coli host strain 5-alpha F′ 1q (New England Biolabs) was exclusively used to generate, store, and amplify the E. coli/B. burgdorferi shuttle vectors listed in Table 2. The resulting strains were grown on LB agar plates or in Super Broth (35 g/L bacto-tryptone, 20 g/L yeast extract, 5 g/L NaCl, and 6 mM NaOH) liquid medium with shaking at 30° C. Transformation was achieved by heat shock followed by recovery in SOC medium (New England Biolabs) for 1h at 30° C. with shaking. Antibiotic selection was achieved using spectinomycin at 50 μg/mL or rifampin at 25 μg/mL in liquid culture or 50 μg/mL in plates.

B. burgdorferi strains and growth conditions: Previously described B. burgdorferi strain B31-A3-68-Δbbe02::P_{flgB}-aphI, also known as K2, is an infectious, highly transformable derivative of the type strain B31. To derive strain CJW_Bb471 from K2, pseudogene bbf29 of plasmid lp28-1 was disrupted by insertion of a gentamicin resistance cassette. Strains K2 and CJW_Bb471 contain 18 of the 21 endogenous plasmids of parental strain B31; they both lack endogenous plasmids cp9, lp5, and lp56. To generate strain CJW_Bb471, 75 μg of plasmid p28-1::flgBp-aacC1 were digested with AgeI-HF (New England Biolabs), ethanol precipitated, resuspended in 25 μL water, and electroporated into a 100 μL aliquot of K2 electrocompetent cells. Electroporated cells were immediately transferred to 6 mL complete Barbour-Stoenner-Kelly (BSK)-II medium and allowed to recover overnight. The following day, cells were plated in semisolid BSK-agarose medium under kanamycin and gentamicin selection. A clone was grown and confirmed to have correct insertion of the gentamicin resistance cassette into lp28-1 and to contain all the endogenous plasmids of the parental strain.

B. burgdorferi strains were grown in complete BSK-II medium at 34° C. in a humidified 5% CO2 incubator. BSK-II medium contained 50 g/L Bovine Serum Albumin, Universal Grade (Millipore), 9.7 g/L CMRL-1066 (US Biological), 5 g/L Neopeptone (Difco), 2 g/L Yeastolate (Difco) 6 g/L HEPES (Millipore), 5 g/L glucose (Sigma-Aldrich), 2.2 g/L sodium bicarbonate (Sigma-Aldrich), 0.8 g/L sodium pyruvate (Sigma-Aldrich), 0.7 g/L sodium citrate (Fisher Scientific), 0.4 g/L N-acetylglucosamine (Sigma-Aldrich), 60 mL/L heat-inactivated rabbit serum (Gibco), and had a pH of 7.6. For plating in semisolid BSK-agarose medium, each 10-cm plate was seeded with up to 1 mL B. burgdorferi culture. BSK-agarose plating medium was made by mixing two volumes of 1.7% agarose in water, melted and pre-equilibrated at 55° C. with three volumes of BSK-1.5 medium, also briefly (for less than 5 min) pre-equilibrated at 55° C. and containing appropriate amounts of antibiotics. Then, 25 mL of the BSK-agarose mix was added to each seeded plate, which was then gently swirled and allowed to solidify for approximately 30 min at room temperature in a biosafety cabinet. The plates were then transferred to a humidified 5% CO2 incubator kept at 34° C. BSK-1.5 medium contained 69.4 g/L BSA, 12.7 g/L CMRL-1066, 6.9 g/L Neopeptone, 3.5 g/L Yeastolate, 8.3 g/L HEPES, 6.9 g/L glucose, 6.4 g/L sodium bicarbonate, 1.1 g/L sodium pyruvate, 1.0 g/L sodium citrate, 0.6 g/L N-acetylglucosamine, 40 mL/L heat-inactivated rabbit serum, and had a pH of 7.5. Antibiotics were used at the following concentrations: streptomycin at 100m/mL, gentamicin at 40 μg/mL, and kanamycin at 200m/mL. Unless otherwise indicated, B. burgdorferi cultures were maintained in exponential growth by diluting cultures into fresh medium before cultures densities reached approximately 5×10 7 cells/mL. Cell density of cultures was determined by direct counting under darkfield illumination using disposable hemocytometers, as previously described in the art.

B. burgdorferi transformation, clone isolation, and characterization. Electrocompetent cells were generated as previously described and stored as single use 50 or 100 μL aliquots at −80° C. For shuttle vector transformations, 30 or 50 μg of plasmid eluted in water were electroporated (2.5 kV, 25 1.4F, 200 Ω, 2 mm gap cuvette) into 50 μL aliquots of competent cells. Electroporated cells were immediately transferred to 6 mL BSK-II and allowed to recover overnight. The next day, 100, 300, and 900 μL aliquots of the culture were each plated in semisolid BSK-agarose under selection. The remaining culture was diluted 6-fold in BSK-II and selected in liquid culture with appropriate antibiotics. Once transformants were observed as motile spirochetes, the liquid cultures were plated for clone isolation. Agarose plugs containing individual colonies were used to inoculate 6 mL BSK-II cultures. After 3 days, 500 to 1000 μL of each clonal culture was removed and pelleted at 10,000×g for 10 min, the cells were resuspended and lysed in 50-100 μL water, and the resulting solution was used to perform multiplex PCR using primer pairs specific for each endogenous plasmid of strain B31 and the DreamTaq™ Green DNA Polymerase (Thermo Scientific). For genomic DNA extraction, approximately 14 mL cultures were grown to approximately 10 8 cells/mL and then pelleted at 4,300×g for 10 min at room temperature in a Beckman Coulter X-14R centrifuge equipped with a swinging bucket rotor. The media was removed and the pellet was processed for DNA extraction using QIAGEN's DNeasy® Blood & Tissue Kit protocol for Gram-negative bacteria. Final elution was carried out in 10 mM Tris-HCl, 0.1 mM EDTA, pH 9.0.

Generation of E. coli/B. burgdorferi shuttle vectors for Cas9 and sgRNA expression: Table 2 lists the E. coli/B. burgdorferi shuttle vectors used or generated in this study. They were based on the previously described B. burgdorferi CRISPR interference platform. The shuttle vectors express one of the following Cas9 versions: wild-type Cas9, the nickases Cas9^{D10A} or Cas9^{H840A}, or the catalytically inactive dCas9 that carries both the D10A and H840A mutations. To revert the D10A mutation, site-directed mutagenesis was performed on appropriate template plasmids using Agilent's QuickChange® Lightning Site-Directed Mutagenesis kit and primers NT651 and NT652. To revert the H840A mutation, site-directed mutagenesis was performed on appropriate template plasmids using primers NT749 and NT750. To generate plasmids with decreased basal expression of Cas9 proteins, site-directed mutagenesis was performed on appropriate plasmid templates using primers NT669 and NT670, which generated a weakened ribosomal binding site (“RB Smut” constructs), or primers NT677 and NT678, which introduced a mutation in the −10 region of the Cas9 promoter (“-10TC” constructs). Expression cassettes for the sgRNAs were moved among plasmids using restriction endonucleases AscI and EagI. To generate sgRNA expression cassettes, SapI-digested Psyn-sgRNA500-containing plasmids were ligated with annealed primer pairs, as follows: primers NT657 and NT658 generated sgRNAvlsE1; NT660 and NT661 generated sgRNAvlsE2; NT721 and NT722 generated sgRNAvls11; NT723 and NT724 generated sgRNAbbe10; NT725 and NT726 generated sgRNAbbe17; and NT727 and NT728 generated sgRNAbbf03. Primer annealing was achieved by mixing 10 μL volumes of each primer at 5 μM concentration, then cycling the mix five times between 30 s at 95° C. and 30 s at 55° C., followed by cooling to room temperature. Nucleotide sequences of primers used to generate the E. coli/B. burgdorferi shuttle vectors in this study are given in Table 6.

DNA sequence analysis: To determine the sequence of the vls locus of B. burgdorferi strain K2, the 10910 base pair region encompassing vlsE and silent cassettes vls2-vls16 was amplified using Platinum™ SuperFi™ DNA Polymerase (Thermo Fisher Scientific) and primers YN-LI_266 and YN-LI_267 (Table 6) and then sequenced with a SMRT Cell™ using 10-h data collection (Pacific Biosciences). The resulting reads were subjected to read-of-insert (ROI) analysis using SMRT Link v6.0.0 (Pacific Biosciences), followed by multiple sequence alignment, to obtain the final consensus sequence.

### Example 1: Expression and Targeting of Cas9 Activity in B. burgdorferi

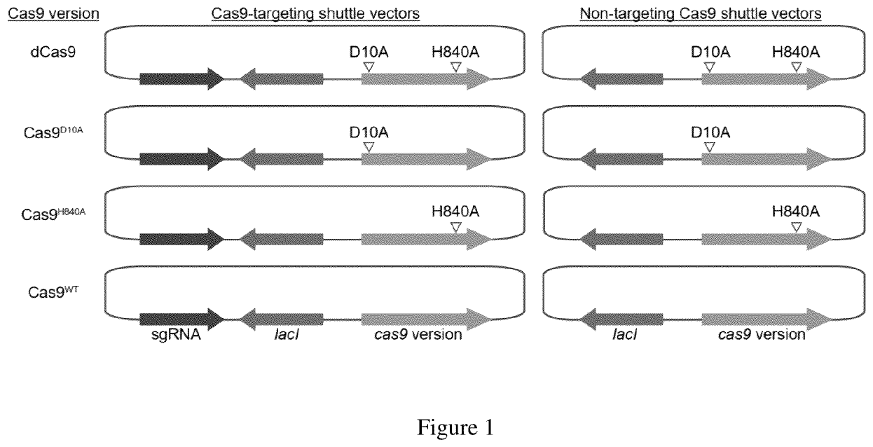

The previous report establishing CRISPRi in B. burgdorferi relied in part on all-in-one E. coli 1 B. burgdorferi shuttle vectors that carry a constitutive sgRNA expression cassette as well as an isopropyl β-D-1-thioglactopyranoside (IPTG)-inducible dCas9 expression cassette. Using these CRISPRi shuttle vectors or control vectors that lack the sgRNA as background, we generated vectors (FIG. 1) that express either Cas9^{WT}, which cleaves both DNA strands, or nickases Cas9^{H840A} and Cas9^{D10A}, which cleave only one DNA strand.

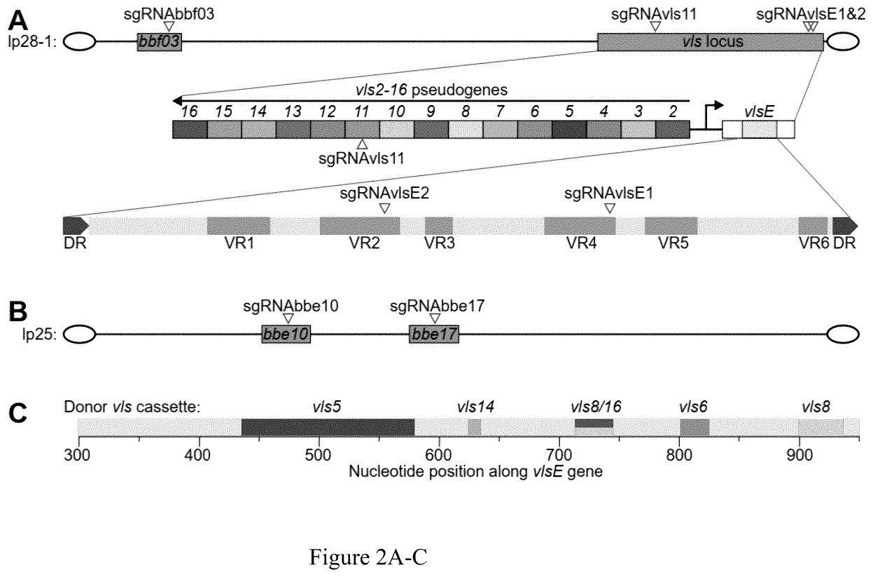

In separate cultures, Cas9^{WT} or its nickase versions were targeted to two endogenous B. burgdorferi plasmids, lp25 and lp28-1 (FIG. 2A-C). Plasmid lp25 encodes the nicotinamidase PncA which is essential for B. burgdorferi's survival in the tick and vertebrate hosts. Plasmid lp28-1 carries the vls antigenic variation system, which is composed of the expressed vlsE lipoprotein gene and 15 silent vls cassettes, vls2-vls16 (FIG. 2A), and is needed for the establishment of persistent infection in immunocompetent vertebrate hosts. For lp28-1, two different sites in vlsE were targeted, plus one site in one of the silent vls cassettes, vls//, and another in the non-vis locus bbf03 (FIG. 2A). For lp25, genes bbe10 and bbe17 were selected and targeted individually (FIG. 2B). The sequences of the spacer and the PAM of these sgRNAs are listed in Table 1.

TABLE 1
sgRNAs used in this study

| | | | | B. |
|---|---|---|---|---|
| | | SEQ | | burgdorferi |
| sgRNA | Guide RNA spacer | ID | | target |
| ID | sequence (5′ to 3′) | NO: | PAM | plasmid |
| bbe10 | AGGGGGAAGACAATTTACTT | 1 | TGG | lp25 |
| bbe17 | AATATTCTTTCAGGGTAAGC | 2 | AGG | lp25 |
| vlsE1 | GGATGGAGAGAAGCCTGAGG | 3 | AGG | lp28-1 |
| vlsE2 | GCTACAGGGGAGAATAATAA | 4 | AGG | lp28-1 |
| vls11 | GCTGTTAGTGCTGGTTAGTG | 5 | TGG | lp28-1 |
| bbf03 | AGAGTTTCTACGATTGAGTA | 6 | TGG | lp28-1 |

For these experiments, strain B31-A3-68 Δbbe02::P_{flgB}-aphI, also known as K2, a transformable, clonal, infectious derivative of the type strain B31, was used. A mouse passage occurred during the derivation of strain K2 from the parental, sequenced B31 strain. During that mouse passage, gene conversion events likely changed the vlsE sequence. The entire vls locus of strain K2 was sequenced using long read single-molecule, real-time (SMRT) sequencing to obtain an accurate sequence encompassing the expressed vlsE gene and the repetitive silent vls cassettes. The sequence of the silent vls cassette region was identical to the B. burgdorferi B31 reference sequence (GenBank accession number AE000794.2). In contrast, the sequence of the vlsE gene of strain K2 had indeed diverged from the parental B31 vlsE, as expected. Five clusters of changes were detected that could be attributed to segmental gene conversion events in which the original sequence was replaced by segments copied from the vls2-vls16 silent cassette sequences (FIG. 2C). Based on the alignment of the K2 vlsE sequence with the silent vls2-16 cassette sequences, two sgRNAs were designed, sgRNAvlsE1 and sgRNAvlsE2, to maximize on-target (vlsE) and minimize off-target (the rest of the genome including vls2-16) binding potential (FIG. 2C and Table 1). A shuttle vector carrying both cas9″ and sgRNAvlsE2 was not active, possibly due to toxicity of DSBs associated with off-target Cas9^{WT} activity in E. coli. Shuttle vectors carrying genes encoding dCas9, cas9^{D10A}, or Cas9^{H840A}, in combination with sgRNAvlsE2 were prepared. The shuttle vectors containing these constructs are listed in Table 2. Takas 2021 refers to Takacs C N, Scott M, Chang Y, Kloos Z A, Irnov I, Rosa P A, et al. A CRISPR interference platform for selective downregulation of gene expression in Borrelia burgdorferi. Appl Environ Microbiol. 2021; 87:e02519-20. doi: 10.1128/AEM.02519-20. PubMed PMID: 33257311; PubMed CentralPMCID: PMCPMC7851697.

TABLE 2
E. coli/B. burgdorferi shuttle vectors^{a} used in this study

| | | | |
|---|---|---|---|
| | CJW strain | | Source or |
| Shuttle vector name | number^{b} | Selection^{c} | Reference |

i. Shuttle vectors expressing catalytically inactive dCas9

| | | | |
|---|---|---|---|
| pBbdCas9S | | Sm/Sp | |
| pBbdCas9S_arr2 | | Sm/Sp, Rf | Takas 2021 |
| pBbdCas9S_P_{syn}-sgRNA500 | | Sm/Sp | Takas 2021 |
| pBbdCas9G_arr2 | | Gm, Rf | Takas 2021 |
| pBbdCas9S(RBSmut) | | Sm/Sp | Takas 2021 |
| pBbdCas9S(RBSmut)_arr2 | | Sm/Sp, Rf | Takas 2021 |
| pBbdCas9S(RBSmut)_P_{syn}-sgRNA500 | | Sm/Sp | |
| pBbdCas9S(RBSmut)_P_{syn}-sgRNAvlsE1 | CJW7267 | Sm/Sp | This study |
| pBbdCas9S(RBSmut)_P_{syn}-sgRNAvlsE2 | CJW7268 | Sm/Sp | This study |
| pBbdCas9S(RBSmut)_P_{syn}-sgRNAvls11 | CJW7269 | Sm/Sp | This study |
| pBbdCas9S(RBSmut)_P_{syn}-sgRNAbbf03 | CJW7282 | Sm/Sp | This study |
| pBbdCas9S(RBSmut)_P_{syn}-sgRNAbbe10 | CJW7280 | Sm/Sp | This study |
| pBbdCas9S(RBSmut)_P_{syn}-sgRNAbbe17 | CJW7281 | Sm/Sp | This study |

ii. Shuttle vectors expressing the nickase Cas9^{D10A}

| | | | |
|---|---|---|---|
| pBbCas9^{D10A}S(RBSmut) | CJW7290 | Sm/Sp | This study |
| pBbCas9^{D10A}S(RBSmut)_arr2 | CJW7291 | Sm/Sp, Rf | This study |
| pBbCas9^{D10A}S(RBSmut)_P_{syn}-sgRNA500 | CJW7292 | Sm/Sp | This study |
| pBbCas9^{D10A}S(RBSmut)_P_{syn}-sgRNAvlsE1 | CJW7293 | Sm/Sp | This study |
| pBbCas9^{D10A}S(RBSmut)_P_{syn}-sgRNAvlsE2 | CJW7294 | Sm/Sp | This study |
| pBbCas9^{D10A}S(RBSmut)_P_{syn}-sgRNAvls11 | CJW7295 | Sm/Sp | This study |
| pBbCas9^{D10A}S(RBSmut)_P_{syn}-sgRNAbbf03 | CJW7298 | Sm/Sp | This study |
| pBbCas9^{D10A}S(RBSmut)_P_{syn}-sgRNAbbe10 | CJW7296 | Sm/Sp | This study |
| pBbCas9^{D10A}S(RBSmut)_P_{syn}-sgRNAbbe17 | CJW7297 | Sm/Sp | This study |

iii. Shuttle vectors expressing the nickase Cas9^{H840A}

| | | | |
|---|---|---|---|
| pBbCas9^{H840A}S | CJW7108 | Sm/Sp | This study |
| pBbCas9^{H840A}S_arr2 | CJW7109 | Sm/Sp, Rf | This study |
| pBbCas9^{H840A}S_P_{syn}-sgRNA500 | CJW7110 | Sm/Sp | This study |
| pBbCas9^{H840A}S_P_{syn}-sgRNAvlsE1 | CJW7128 | Sm/Sp | This study |
| pBbCas9^{H840A}S_P_{syn}-sgRNAvlsE2 | CJW7129 | Sm/Sp | This study |
| pBbCas9^{H840A}S_P_{syn}-sgRNAvls11 | CJW7246 | Sm/Sp | This study |
| pBbCas9^{H840A}S_P_{syn}-sgRNAbbf03 | CJW7249 | Sm/Sp | This study |
| pBbCas9^{H840A}S_P_{syn}-sgRNAbbe10 | CJW7247 | Sm/Sp | This study |
| pBbCas9^{H840A}S_P_{syn}-sgRNAbbe17 | CJW7248 | Sm/Sp | This study |
| pBbCas9^{H840A}S(RBSmut) | CJW7155 | Sm/Sp | This study |
| pBbCas9^{H840A}S(RBSmut)_arr2 | CJW7166 | Sm/Sp, Rf | This study |
| pBbCas9^{H840A}S(RBSmut)_P_{syn}-sgRNA500 | CJW7157 | Sm/Sp | This study |
| pBbCas9^{H840A}S(RBSmut)_P_{syn}-sgRNAvlsE1 | CJW7158 | Sm/Sp | This study |
| pBbCas9^{H840A}S(RBSmut)_P_{syn}-sgRNAvlsE2 | CJW7159 | Sm/Sp | This study |
| pBbCas9^{H840A}S(RBSmut)_P_{syn}-sgRNAvls11 | CJW7250 | Sm/Sp | This study |
| pBbCas9^{H840A}S(RBSmut)_P_{syn}-sgRNAbbf03 | CJW7253 | Sm/Sp | This study |
| pBbCas9^{H840A}S(RBSmut)_P_{syn}-sgRNAbbe10 | CJW7251 | Sm/Sp | This study |
| pBbCas9^{H840A}S(RBSmut)_P_{syn}-sgRNAbbe17 | CJW7252 | Sm/Sp | This study |
| pBbCas9^{H840A}S(−10 TC) | CJW7160 | Sm/Sp | This study |
| pBbCas9^{H840A}S(−10 TC)_arr2 | CJW7161 | Sm/Sp, Rf | This study |
| pBbCas9^{H840A}S(−10 TC)_P_{syn}-sgRNA500 | CJW7162 | Sm/Sp | This study |
| pBbCas9^{H840A}S(−10 TC)_P_{syn}-sgRNAvlsE1 | CJW7163 | Sm/Sp | This study |
| pBbCas9^{H840A}S(−10 TC)_P_{syn}-sgRNAvlsE2 | CJW7164 | Sm/Sp | This study |
| pBbCas9^{H840A}S(−10 TC)_P_{syn}-sgRNAvls11 | CJW7254 | Sm/Sp | This study |
| pBbCas9^{H840A}S(−10 TC)_P_{syn}-sgRNAbbe17 | CJW7255 | Sm/Sp | This study |

iv. Shuttle vectors expressing wild-type Cas9

| | | | |
|---|---|---|---|
| pBbCas9S(RBSmut) | CJW7283 | Sm/Sp | This study |
| pBbCas9S(RBSmut)_arr2 | CJW7284 | Sm/Sp, Rf | This study |
| pBbCas9S(RBSmut)_P_{syn}-sgRNA500 | CJW7285 | Sm/Sp | This study |
| pBbCas9S(RBSmut)_P_{syn}-sgRNAvlsE1 | CJW7286 | Sm/Sp | This study |
| pBbCas9S(RBSmut)_P_{syn}-sgRNAvls11 | CJW7278 | Sm/Sp | This study |
| pBbCas9S(RBSmut)_P_{syn}-sgRNAbbf03 | CJW7279 | Sm/Sp | This study |
| pBbCas9S(RBSmut)_P_{syn}-sgRNAbbe10 | CJW7288 | Sm/Sp | This study |
| pBbCas9S(RBSmut)_P_{syn}-sgRNAbbe17 | CJW7289 | Sm/Sp | This study |

^{a}Naming of the E. coli/B. burgdorferi shuttle vectors follows the nomenclature established and described in detail in Takas 2012. Of note, Cas9 variant expression is driven either by the IPTG-inducible P_{pQE30} promoter or by its mutant versions in which the −10 region of the promoter (−10 TC) or the ribosome binding site (RBSmut) were mutated to reduce basal Cas9 expression;
^{b}When requesting a plasmid from the Jacobs- Wagner lab, please include the CJW strain number alongside the plasmid name. For constructs previously published in Takas 2012, a CJW strain number is not provided, as the plasmids are available from Addgene. Refer to the original publication for the Addgene catalog numbers;
^{c}Sm/Sp, streptomycin/spectinomycin resistance conferred by the aadA gene; Rf, rifampin resistance conferred by the arr2 gene; Gm, gentamicin resistance conferred by the aacC1 gene.

### Example 2: Targeting Cas9 Activity to Endogenous B. burgdorferi Plasmids Causes Plasmid Loss

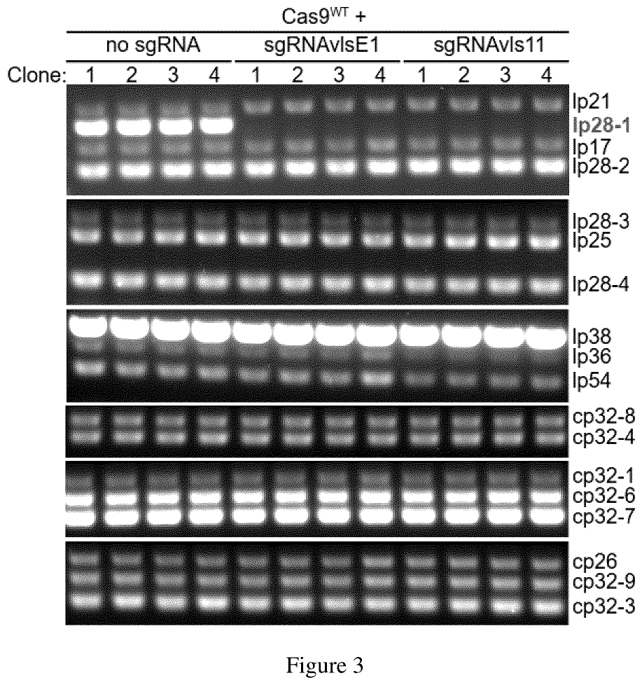

The shuttle vectors described above were electroporated into strain K2. As controls, shuttle vectors lacking the sgRNA cassette and shuttle vectors expressing dCas9 rather than Cas9^{WT} were used (Table 2). For each construct, the electroporated cells were plated after about three generations, a small number of the resulting clones were grown, and their endogenous plasmid content was determined by multiplex PCR, as described in the art. All clones that had received a shuttle vector expressing Cas9^{WT} and the vlsE-targeting sgRNAvlsE1 had lost the vlsE-carrying plasmid lp28-1 (Table 3, FIG. 3). This was not due to widespread loss of lp28-1 from the parental strain, as clones obtained from electroporation of a shuttle vector expressing Cas9^{WT} but no sgRNA retained their lp28-1 plasmid (Table 3, FIG. 3). Similarly, electroporation of shuttle vectors encoding catalytically inactive dCas9, either alone or alongside sgRNAvlsE1 or sgRNAvlsE2, did not cause widespread lp28-1 loss (Table 3). The loss of lp28-1 occurred in spite of the presence of the adjacent homologous vls2-vls16 sequences that are used as donors for the generation of variant vlsE sequences during mammalian infection. There was also extensive plasmid loss when Cas9^{WT} was targeted to two other sites on lp28-1: the silent cassette vls11 or to the non-vls gene bbf03 (Table 3, FIG. 3). Therefore, Cas9^{WT}-mediated lp28-1 loss requires both Cas9 activity and targeting of this activity to the lp28-1 plasmid by a sgRNA regardless of where the DNA cut occurs. This effect was not limited to lp28-1, as targeting Cas9^{WT} to genes bbe10 or bbe17 on endogenous plasmid lp25 resulted in loss of plasmid lp25 but not of lp28-1 (Table 3). All other endogenous B. burgdorferi plasmids were retained in almost all clones analyzed (Table 3). As with lp28-1, the loss of lp25 was dependent on Cas9 activity and the expression of a lp25-specific sgRNA, as expressing Cas9^{WT} alone, or targeting dCas9 to lp25 did not affect lp25 retention (Table 3). We note that the transformants were selected and grown in the absence of Cas9 expression by IPTG induction. Presumably, the previously documented low but detectable basal expression of Cas9 from this system generates enough activity to induce plasmid loss.

TABLE 3
Endogenous plasmid retention in individual B. burgdorferi clones after
electroporation of Cas9/sgRNA vectors, as detected by multiplex PCR^{b}

| | Endogenous | | | Plasmid retention (detected count/ | |
|---|---|---|---|---|---|
| | B. burgdorferi | | | expected count for full retention)^{b} | |
| Cas9 | plasmid | | Clones | Targeted plasmid | All other tested |
| version | targeted | sgRNA | analyzed | (lp28-1 or lp25) | plasmids combined |
| Cas9^{WT} | None | None | 4 | N/A^{c} | 72/72 |
| | lp28-1 | vlsE1 | 4 | 0/4 | 68/68 |
| | | vls11 | 4 | 0/4 | 68/68 |
| | | bbf03 | 4 | 0/4 | 68/68 |
| | lp25 | bbe10 | 4 | 0/4 | 67/68 |
| | | bbe17 | 4 | 0/4 | 68/68 |
| dCas9 | None | None | 4 | N/A | 72/72 |
| | lp28-1 | vlsE1 | 4 | 4/4 | 68/68 |
| | | vlsE2 | 4 | 3/4 | 67/68 |
| | | vls11 | 4 | 4/4 | 68/68 |
| | | bbf03 | 4 | 4/4 | 68/68 |
| | lp25 | bbe10 | 4 | 4/4 | 68/68 |
| | | bbe17 | 4 | 4/4 | 68/68 |
| | None | None | 4 | N/A | 70/72 |
| | | vlsE1 | 4 | 0/4 | 68/68 |
| | | vlsE2 | 4 | 0/4 | 68/68 |
| Cas9^{D10A} | lp28-1 | vls11 | 4 | 0/4 | 68/68 |
| | | bbf03 | 4 | 3/4 | 68/68 |
| | lp25 | bbe10 | 4 | 0/4 | 68/68 |
| | | bbe17 | 4 | 4/4 | 68/68 |
| Cas9^{H840A} | None | None | 8 | N/A | 143/144 |
| | lp28-1 | vlsE1 | 16 | 0/16 | 272/272 |
| | | vlsE2 | 16 | 0/16 | 272/272 |
| | | vls11 | 4 | 0/4 | 68/68 |
| | | bbf03 | 4 | 2/4 | 68/68 |
| | lp25 | bbe10 | 4 | 0/4 | 68/68 |
| | | bbe17 | 4 | 4/4 | 68/68 |

^{b}Data was aggregated based on the Cas9 version and the sgRNA expressed by the shuttle vector. Transformed strains carrying the same sgRNA but expressing different basal levels of the Cas9 variant were analyzed together. Plasmid detection was achieved by multiplex PCR;
^{b}Data compares the number of endogenous plasmids detected in the analyzed clones with the expected number of endogenous plasmids if they had all been retained. All plasmid counts are combined for the non- targeted plasmids. A total of 18 non-targeted plasmids were assayed for each clone obtained by transformation with a shuttle vector lacking a sgRNA. A total of 17 non-targeted plasmids wereassayed for each clone obtained by transformation with a shuttle vector expressing a sgRNA;
^{c}N/A, not applicable.

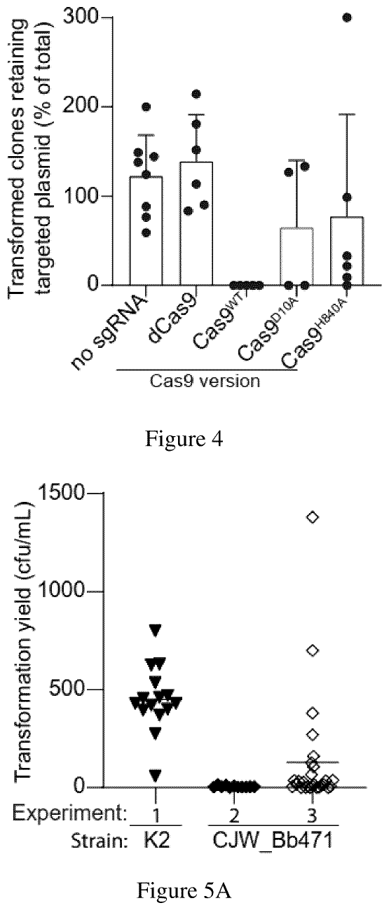

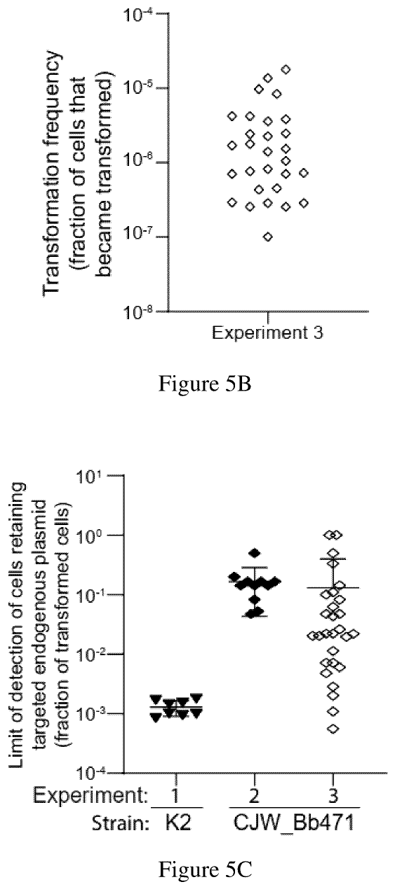

Performing multiplex PCR assays on individual clones is relatively labor-intensive. Additionally, if Cas9^{WT}-mediated plasmid loss is not 100% effective, the fraction of cells that still retain the targeted plasmid might be below detection. To avoid these drawbacks, endogenous plasmid retention was quantified by plating electroporated B. burgdorferi populations under differential antibiotic selection. In these plating assays, strain K2 was used, in which retention of plasmid lp25 allows colony formation in the presence of kanamycin. Additionally, strain CJW Bb471 was derived from strain K2 by inserting a gentamicin resistance cassette in its lp28-1 plasmid. This genetic modification does not interfere with B. burgdorferi's ability to infect mice or be acquired by ticks. Plating CJW Bb471 transformants in the presence of kanamycin assays retention of lp25, while plating in the presence of gentamicin examines retention of lp28-1. In both cases, acquisition of streptomycin resistance indicates successful delivery of the Cas9-expressing shuttle vector. The number of streptomycin-resistant transformants detected in these experiments varied significantly both within an experiment and between experiments (FIG. 5A), as did transformation frequencies, as measured in one of the experiments (FIG. 5B). Despite these limitations, the following trends were detected (see Tables 4 and 5 and FIG. 4). First, targeting Cas9^{WT} to either lp25 or lp28-1 did not result in the recovery of transformants retaining the targeted plasmid. Second, targeted dCas9 did not destabilize the targeted plasmids. Third, all Cas9 versions failed to destabilize lp25 or lp28-1 in the absence of a targeting sgRNA. Based on the number of clones that received the Cas9-expressing shuttle vector in each of the electroporations, we calculated the lowest frequency at which we could detect clones retaining the targeted endogenous plasmids (FIG. 5A-C). These experimental limits of detection of plasmid retention within the transformed cell populations also varied significantly from electroporation to electroporation. The lowest limit of detection was around 10⁻³ (FIG. 5C). This value indicates that DSB repair mechanisms that occur less frequently than in one cell out of 1,000 cells could not be detected in the assay.

TABLE 4
Linear plasmid 25 (lp25) retention in B. burgdorferi
following Cas9 targeting, as detected by plating.

| Host strain | | | Transformants detected by plating (CFU/mL)^{d} | |
|---|---|---|---|---|
| (marked | | | CFU/mL (selection | CFU/mL (selection |
| endogenous | Cas9 | | for shuttle vector and | for shuttle |
| plasmid) | version | sgRNA | endogenous plasmid) | vector alone) |
| K2 (lp25) | Cas9^{WT} | bbe10 | 0 | 910 |
| Experiment 1 | | bbe17 | 0 | 500 |
| | dCas9 | bbe10 | 610 | 760 |
| | | bbe17 | 530 | 450 |
| | Cas9^{D10A} | bbe10 | 118 | 810 |
| | | bbe17 | 370 | 530 |
| | Cas9^{H840A} | bbe10 | 310 | 770 |
| | | bbe17 | 520 | 430 |
| CJW_Bb471 | Cas9^{WT} | None | 10 | 6.9 |
| (lp25) | | bbe10 | 0 | 12.3 |
| Experiment 3^{e} | | bbe17 | 0 | 17.7 |
| | dCas9 | None | 22.3 | 37.7 |
| | | bbe10 | 145 | 127.5 |
| | | bbe17 | 102.5 | 67.5 |
| | Cas9^{D10A} | None | 82.5 | 107.5 |
| | | bbe17 | 202.5 | 160 |
| | Cas9^{H840A} | None | 47.7 | 34.6 |
| | | bbe10 | 0 | 1.5 |
| | | bbe17 | 1360 | 1380 |

^{d}Different volumes of transformant cultures were plated under streptomycin selection (which selects for the shuttle vector), or streptomycin + kanamycin selection (which selects for lp25). Colonies were counted after 2-3 weeks and the resulting count was used to calculate the concentration of selectable cells in the parental population of transformants, expressed as colony forming units (CFU) per mL;
^{e}Retention of both lp25 and lp28-1 was assayed in experiment 3 following electroporation of the indicated constructs. For this reason, results from this experiment are presented in both Tables 4 and 5.

TABLE 5
Linear plasmid 28-1 (lp28-1) retention in B. burgdorferi
following Cas9 targeting, as detected by plating.

| Host strain | | | Transformants detected by plating (CFU/mL)^{f} | |
|---|---|---|---|---|
| (marked | | | CFU/mL (selection for | CFU/mL (selection |
| endogenous | Cas9 | | shuttle vector and | for shuttle |
| plasmid) | version | sgRNA | endogenous plasmid) | vector alone) |
| CJW_Bb471 | Cas9^{WT} | None | 6.9 | 16.2 |
| (lp28-1) | | vlsE1 | 0 | 5.4 |
| Experiment 2 | dCas9 | None | 2.3 | 4.6 |
| | | vlsE1 | 6.9 | 4.6 |
| | | vlsE2 | 3.1 | 9.2 |
| | Cas9^{D10A} | None | 2.3 | 1.5 |
| | | vlsE1 | 1.5 | 5.4 |
| | | vlsE2 | 3.1 | 4.6 |
| | Cas9^{H840A} | None | 0.8 | 3.8 |
| | | vlsE1 | 0 | 14.6 |
| | | vlsE2 | 0 | 5.4 |
| CJW_Bb471 | Cas9^{WT} | None | 13.8 | 6.9 |
| (lp28-1) | | vlsE1 | 0 | 380 |
| Experiment 3^{g} | | vls11 | 0 | 16.1 |
| | | bbf03 | 0 | 29.2 |
| | dCas9 | None | 46.9 | 37.7 |
| | | vlsE1 | 35.4 | 39.2 |
| | | vlsE2 | 7.7 | 9.2 |
| | | vls11 | 29.2 | 16.1 |
| | | bbf03 | 11.5 | 5.4 |
| | Cas9^{D10A} | None | 95 | 107.5 |
| | | vlsE2 | 0 | 7.7 |
| | | vls11 | 0 | 0.8 |
| | | bbf03 | 3.1 | 2.3 |
| | Cas9^{H840A} | None | 51.5 | 34.6 |
| | | vlsE1 | 64.6 | 700 |
| | | vlsE2 | 58.5 | 270 |
| | | vls11 | 11.5 | 35 |
| | | bbf03 | 2.3 | 0.8 |

^{f}Different volumes of transformant cultures were plated under streptomycin selection (which selects for the shuttle vector), or streptomycin + gentamicin (which selects for lp28-1). Colonies were counted and the resulting count was used to calculate the concentration of selectable cells in the parental population of transformants, expressed as colony forming units (CFU) per mL;
^{g}Retention of both lp25 and lp28-1 was assayed in experiment 3 following electroporation of the indicated constructs. For this reason, results from this experiment are presented in both Tables 4 and 5.

### Example 3: Destabilizing Effects of Cas9 Nickases on B. burgdorferi Endogenous Plasmids

While Cas9^{WT} robustly and specifically induced plasmid loss when targeted to lp25 or lp28-1 (Tables 3-5, FIG. 4), the nickases Cas9^{D10A} and Cas9^{H840A} exhibited more heterogeneous behaviors. When analyzing by multiplex PCR the clones isolated in the absence of selection for the targeted plasmid, targeting the nickases to the vls region of lp28-1 or the bbe10 locus of lp25 was more efficient at causing plasmid loss than targeting the nickases to the bbf03 locus of lp28-1 or the bbe17 locus of lp25 (Table 3). A similar trend was observed when the transformants were selected for the targeted plasmid (Tables 4 and 5, FIG. 4). These differences could be due to distinct targeting efficiencies by the sgRNAs or could reflect varied efficiencies in repairing SSBs induced at the sgRNA-targeted location.

TABLE 6
Oligonucleotide primers

| Name | SEQ ID NO: | Sequence (5′ to 3′) |
|---|---|---|
| NT651 | 7 | GGATAAGAAATACTCAATAGGCTTAGATATCGGCACAAATA |
| | | GCGTCGGATGGG |
| NT652 | 8 | CCCATCCGACGCTATTTGTGCCGATATCTAAGCCTATTGAGT |
| | | ATTTCTTATCC |
| NT657 | 9 | AGTGCTACAGGGGAGAATAATAA |
| NT658 | 10 | AACTTATTATTCTCCCCTGTAGC |
| NT660 | 11 | AGTGGATGGAGAGAAGCCTGAGG |
| NT661 | 12 | AACCCTCAGGCTTCTCTCCATCC |
| NT669 | 13 | GATAACAATTTCACACAGAATTCATTAAAGAAGAGAAATTA |
| | | CATATGGATAAGAAATAC |
| NT670 | 14 | GTATTTCTTATCCATATGTAATTTCTCTTCTTTAATGAATTCT |
| | | GTGTGAAATTGTTATC |
| NT677 | 15 | GCTTTGTGAGCGGATAACAATTATAACAGATTCAATTGTGAG |
| | | CGGATAACAATTTCACAC |
| NT678 | 16 | GTGTGAAATTGTTATCCGCTCACAATTGAATCTGTTATAATT |
| | | GTTATCCGCTCACAAAGC |
| NT721 | 17 | AGTGCTGTTAGTGCTGGTTAGTG |
| NT722 | 18 | AACCACTAACCAGCACTAACAGC |
| NT723 | 19 | AGTAGGGGGAAGACAATTTACTT |
| NT724 | 20 | AACAAGTAAATTGTCTTCCCCCT |
| NT725 | 21 | AGTAATATTCTTTCAGGGTAAGC |
| NT726 | 22 | AACGCTTACCCTGAAAGAATATT |
| NT727 | 23 | AGTAGAGTTTCTACGATTGAGTA |
| NT728 | 24 | AACTACTCAATCGTAGAAACTCT |
| NT749 | 25 | TAATCGTTTAAGTGATTATGATGTCGATCATATTGTTCCACA |
| | | AAGTTTCCTTAAAGACG |
| NT750 | 26 | CGTCTTTAAGGAAACTTTGTGGAACAATATGATCGACATCAT |
| | | AATCACTTAAACGATTA |
| YN-LI_266 | 27 | GTATTTGTTGTTAAGTAGATAGGAATATTTCGG |
| YN-LI_267 | 28 | CGTGTCCATACACTTAATTAAATCACTTATTC |

### Example 4: Rapid and Timely Cas9-Targeting to Virulence Plasmids in Animals Using the Improved Cas9 Construct

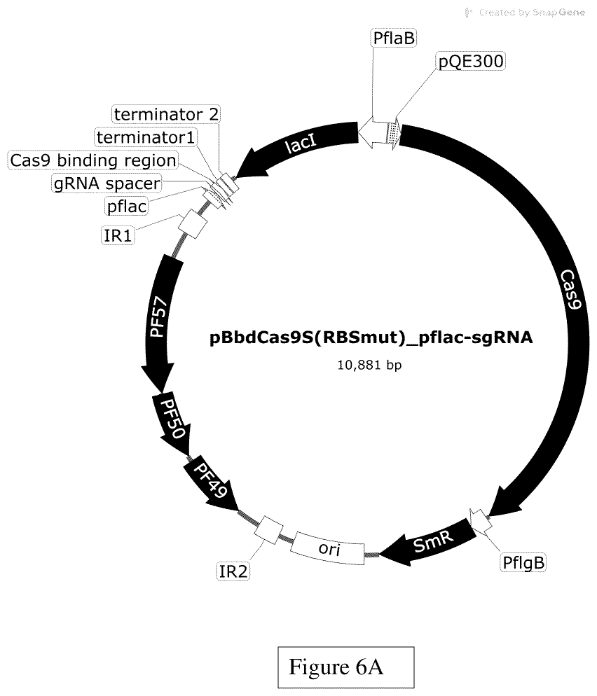

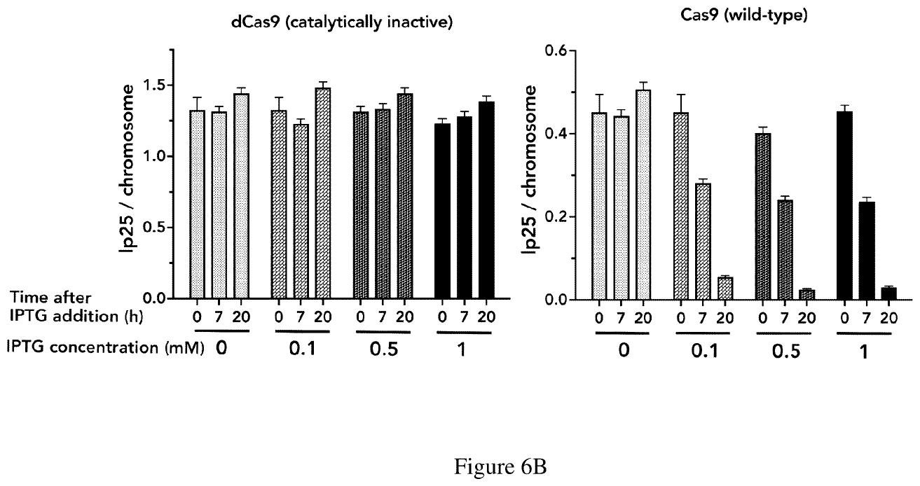

Due to constitutive Cas9-targeting, the use of the original Cas9 construct (Table 2, FIG. 1) is limited to cultured Borrelia. The new construct (FIG. 6A) allows conditional Cas9-targeting to virulence plasmids or chromosomes in Borrelia infecting animals. This was done by replacing the synthetic constitutive promoter that drives an sgRNA in the original constructs with another IPTG-responsible promoter. This construct induced rapid and selective plasmid elimination at a low level of Cas9-sgRNA expression (0.1 mM inducer IPTG). In contrast, the strain containing catalytically inactive Cas9 (dCas9) retained the targeted plasmid at the end of the time course. See FIG. 6B. Similar to the original construct, all non-targeted endogenous plasmids were retained in both strains. IPTG is added to the drinking water for the experimental animals at the time of Cas9-targeting.

### Discussion

Targeting dCas9 to selected B. burgdorferi genes causes specific and efficient downregulation of gene expression, allowing for relatively easy and fast strain generation and phenotypic investigation. As shown herein, targeting Cas9^{WT} or its nickase variants to plasmid-encoded loci results in plasmid loss, though to a varying degree (Tables 3-5 and FIG. 4). In the case of Cas9^{WT}, plasmid loss was very efficient, indicating that repair of double-stranded DNA breaks generated in this manner occurs below the detection limit of our assay, i.e., less than one in 10 3 cells retained the targeted endogenous plasmid, based on the highest number of transformants recovered after Cas9 shuttle vector electroporation (FIG. 5A,C). The nickases Cas9^{D10A} and Cas9H840A also cause significant plasmid loss. Presumably, a considerable fraction of nicked plasmids undergo degradation before DNA repair factors can be recruited to the site of the SSBs. Alternatively, repair of DNA lesions may be less efficient in B. burgdorferi compared to other bacteria, as several DNA repair factors (e.g., mutH, lexA, ruvC, sbcB, recFOR, recX) are absent from the B. burgdorferi genome. When considering the limit of detection of the assay (FIG. 5C), the results suggest that the efficiency of DSB repairs in B. burgdorferi is at least below 10⁻³ even when donor sequences are present as in the case of vlsE and vls11.

Importantly, targeting Cas9^{WT} to an endogenous B. burgdorferi plasmid is an easy and efficient method to displace the plasmid. The Cas9 nickases can also be used to achieve this outcome, but they are less effective. The Cas9-based approach provides an alternative to the previously developed method that displaces endogenous plasmids through introduction of shuttle vectors belonging to the same plasmid compatibility class. Both methods yield clones in which the targeted endogenous plasmid is replaced by a shuttle vector that carries an antibiotic resistance marker. The Cas9-based method, however, does not require prior knowledge of the targeted plasmid's replication and segregation locus, and involves only an easy cloning step to insert the sgRNA sequence into the Cas9 shuttle vector. Additionally, as Cas9 activity can be simultaneously targeted to multiple locations in the genome by co-expression of relevant sgRNAs, simultaneous removal of multiple plasmids from a B. burgdorferi strain should be achievable via a single transformation.

While the degree of genome segmentation in Borreliaceae is the highest among the known bacteria, other bacteria have segmented genomes that can include circular and linear chromosomes, chromids, megaplasmids, as well as smaller plasmids. Plasmids often encode virulence factors or antibiotic resistance genes and are stably maintained by highly effective plasmid segregation mechanisms that ensure faithful inheritance by daughter cells over generations. The study of plasmid-encoded functions in bacteria other than the Lyme disease spirochetes can therefore be facilitated by implementation of a Cas9-mediated plasmid curation protocol. Translation of this approach across bacterial phyla is likely feasible, as demonstrated by the successful broad implementation of CRISPR-based methods of gene regulation.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

What is claimed is:

1. A method of reducing virulence of a bacterial species with a segmented genome which has infected a mammalian host or a mammalian host cell, comprising
exposing the bacterial genome to an RNA-guided nuclease and a guide RNA (gRNA) and generating a double-stranded or single-stranded break in the bacterial genome, wherein the gRNA base pairs with a target sequence in the bacterial genome.

2. The method of claim 1, wherein the target sequence in the bacterial genome is in a bacterial chromosome or an endogenous plasmid.

3. The method of claim 2, wherein the endogenous plasmid is an endogenous virulence plasmid.

4. The method of claim 1, wherein the bacterial species with a segmented genome is Borrelia sp., Vibrio sp., Agrobacteria sp., Bacillus sp., Brucella sp., Burkholderia sp., Leptospira sp., Rhizobium sp., or Rhodobacteria sp.

5. The method of claim 1, wherein the bacterial species with a segmented genome is a Borrelia sp.

6. The method of claim 5, wherein the Borrelia sp. is Borrelia burgdorferi, Borrelia afzelii, Borrelia garinii, Borrelia hermsii, Borrelia turicatae, or Borrelia parkeri.

7. The method of claim 1, wherein the RNA-guided nuclease is a Class 2 CRISPR nuclease.

8. The method of claim 7, wherein the Class 2 CRISPR nuclease is a Cas9 or Cpf1 nuclease.

9. The method of claim 8, wherein the Cas9 nuclease is a wild type Cas9, Cas9^{D10A}, or Cas9^{H840A}.

10. The method of claim 1, wherein the endogenous virulence plasmid is an lp25, lp28-1, lp36 or cp32 plasmid.

11. The method of claim 10, wherein the plasmid is the lp25 plasmid and the sgRNA base-pairs with a bbe10 or bbe17 gene.

12. The method of claim 11, wherein the spacer sequence of the gRNA comprises SEQ ID NO: 1 or SEQ ID NO: 2.

13. The method of claim 12, wherein the plasmid is the lp28-1 plasmid and the gRNA base-pairs with the vlsE lipoprotein gene, a vls2-vls16 silent cassette, or bbf03.

14. The method of claim 13, wherein the spacer sequence of the gRNA comprises any of SEQ ID NOs: 3-6.

15. The method of claim 1, wherein a gene encoding the RNA-guided nuclease and an expression cassette for the gRNA are carried on a shuttle vector comprising the expression cassette for expressing the gRNA and an inducible expression cassette for expressing the RNA-guided nuclease, wherein the shuttle vector can be replicated in E. coli and the bacterial species with a segmented genome.

16. The method of claim 1, wherein a gene encoding the RNA-guided nuclease and an expression cassette for the gRNA are carried on a shuttle vector comprising the inducible expression cassette for expressing both the gRNA and the RNA-guided nucleases, whereby the efficacy of Cas9-targeting can be tested in mammalian subject.

17. The method of claim 1, wherein an inducible expression cassette for expressing the RNA-guided nuclease and an expression cassette for the gRNA are encoded in the DNA of a bacteriophage.

18. The method of claim 1, wherein the RNA-guided nuclease and gRNA are pre-assembled in a liposome, nanoparticle, or extracellular-vesicle delivery system.

19. The method of claim 1, wherein exposing the bacterial genome comprises administering RNA-guided nuclease and a gRNA to the mammalian subject.

20. The method of claim 20, wherein the mammalian subject is a human or rodent.

21. The method of claim 20, further comprising administering an antibiotic to the subject.


SEQUENCE LISTING (37 sequences)

SEQ ID NO: 1 - RNA, 20 nt, synthetic construct
  source 1..20: mol_type = other RNA
agggggaagacaatttactt

SEQ ID NO: 2 - RNA, 20 nt, synthetic construct
  source 1..20: mol_type = other RNA
aatattctttcagggtaagc

SEQ ID NO: 3 - RNA, 20 nt, synthetic construct
  source 1..20: mol_type = other RNA
ggatggagagaagcctgagg

SEQ ID NO: 4 - RNA, 20 nt, synthetic construct
  source 1..20: mol_type = other RNA
gctacaggggagaataataa

SEQ ID NO: 5 - RNA, 20 nt, synthetic construct
  source 1..20: mol_type = other RNA
gctgttagtgctggttagtg

SEQ ID NO: 6 - RNA, 20 nt, synthetic construct
  source 1..20: mol_type = other RNA
agagtttctacgattgagta

SEQ ID NO: 7 - DNA, 53 bp, synthetic construct
  source 1..53: mol_type = other DNA; note = Primer NT651
ggataagaaatactcaataggcttagatatcggcacaaatagcgtcggatggg

SEQ ID NO: 8 - DNA, 53 bp, synthetic construct
  source 1..53: mol_type = other DNA; note = Primer NT652
cccatccgacgctatttgtgccgatatctaagcctattgagtatttcttatcc

SEQ ID NO: 9 - DNA, 23 bp, synthetic construct
  source 1..23: mol_type = other DNA; note = Primer NT657
agtgctacaggggagaataataa

SEQ ID NO: 10 - DNA, 23 bp, synthetic construct
  source 1..23: mol_type = other DNA; note = Primer NT658
aacttattattctcccctgtagc

SEQ ID NO: 11 - DNA, 23 bp, synthetic construct
  source 1..23: mol_type = other DNA; note = Primer NT660
agtggatggagagaagcctgagg

SEQ ID NO: 12 - DNA, 23 bp, synthetic construct
  source 1..23: mol_type = other DNA; note = Primer NT661
aaccctcaggcttctctccatcc

SEQ ID NO: 13 - DNA, 59 bp, synthetic construct
  source 1..59: mol_type = other DNA; note = Primer NT669
gataacaatttcacacagaattcattaaagaagagaaattacatatggataagaaatac

SEQ ID NO: 14 - DNA, 59 bp, synthetic construct
  source 1..59: mol_type = other DNA; note = Primer NT670
gtatttcttatccatatgtaatttctcttctttaatgaattctgtgtgaaattgttatc

SEQ ID NO: 15 - DNA, 60 bp, synthetic construct
  source 1..60: mol_type = other DNA; note = Primer NT677
gctttgtgagcggataacaattataacagattcaattgtgagcggataacaatttcacac

SEQ ID NO: 16 - DNA, 60 bp, synthetic construct
  source 1..60: mol_type = other DNA; note = Primer NT678
gtgtgaaattgttatccgctcacaattgaatctgttataattgttatccgctcacaaagc

SEQ ID NO: 17 - DNA, 23 bp, synthetic construct
  source 1..23: mol_type = other DNA; note = NT721
agtgctgttagtgctggttagtg

SEQ ID NO: 18 - DNA, 23 bp, synthetic construct
  source 1..23: mol_type = other DNA; note = Primer NT722
aaccactaaccagcactaacagc

SEQ ID NO: 19 - DNA, 23 bp, synthetic construct
  source 1..23: mol_type = other DNA; note = Primer NT723
agtagggggaagacaatttactt

SEQ ID NO: 20 - DNA, 23 bp, synthetic construct
  source 1..23: mol_type = other DNA; note = Primer NT724
aacaagtaaattgtcttccccct

SEQ ID NO: 21 - DNA, 23 bp, synthetic construct
  source 1..23: mol_type = other DNA; note = Primer NT725
agtaatattctttcagggtaagc

SEQ ID NO: 22 - DNA, 23 bp, synthetic construct
  source 1..23: mol_type = other DNA; note = Primer NT726
aacgcttaccctgaaagaatatt

SEQ ID NO: 23 - DNA, 23 bp, synthetic construct
  source 1..23: mol_type = other DNA; note = Primer NT727
agtagagtttctacgattgagta

SEQ ID NO: 24 - DNA, 23 bp, synthetic construct
  source 1..23: mol_type = other DNA; note = Primer NT728
aactactcaatcgtagaaactct

SEQ ID NO: 25 - DNA, 59 bp, synthetic construct
  source 1..59: mol_type = other DNA; note = Primer NT749
taatcgtttaagtgattatgatgtcgatcatattgttccacaaagtttccttaaagacg

SEQ ID NO: 26 - DNA, 59 bp, synthetic construct
  source 1..59: mol_type = other DNA; note = Primer NT750
cgtctttaaggaaactttgtggaacaatatgatcgacatcataatcacttaaacgatta

SEQ ID NO: 27 - DNA, 33 bp, synthetic construct
  source 1..33: mol_type = other DNA; note = Primer YN-LI_266
gtatttgttgttaagtagataggaatatttcgg

SEQ ID NO: 28 - DNA, 32 bp, synthetic construct
  source 1..32: mol_type = other DNA; note = Primer YN-LI_267
cgtgtccatacacttaattaaatcacttattc

SEQ ID NO: 29 - protein, 1345 aa, Streptococcus mutans
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 30 - protein, 1368 aa, Streptococcus pyogenes [sequence omitted]

SEQ ID NO: 31 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 32 - protein, 1334 aa, Listeria innocua
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 33 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 34 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 35 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 36 - protein, 1228 aa, unidentified
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 37 - protein, 1203 aa, unidentified
  source 1..1203: note = Lachnospiraceae bacterium MA2020
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQT